(19) United States
(12) Patent Application Publication
(10) Pub. No.: US 2008/0077431 A1
(43) Pub. Date: Mar. 27, 2008

(54) DIGITAL COMMUNICATION AND MONITORING SYSTEM FOR PATIENTS AND DOCTORS

(76) Inventors: William J. Calder, Chesterfield, VA (US); Joanne Cunningham, Mechanicsville, VA (US); A. Brooks Hollar, Richmond, VA (US); Brandon Saunders, Chester, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/534,887

(22) Filed: Sep. 25, 2006

(51) Int. Cl. G06Q 10/00; A61B 5/00

(52) U.S. Cl. 705/2; 600/300

(57) ABSTRACT

A patient healthcare and monitoring system facilitates communications between doctors and patients. The system includes a server hosting a database containing patient information including patient medical histories. Physicians are provided with a doctor client in the form of, for example, a Tablet PC with which a physician can set up patient alerts, surveys and messages and receive reports, messages and patient history. Patients are provided with a patient client in the form of, for example, a Smart phone, Pocket PC or PDA with which a patient receives alerts, messages and surveys generated by their physician and transmit alert completions, messages and survey answers. The doctor clients and the patient clients communicate with the server so that patients are alerted when to perform prescribed procedures or take prescribed medications and physicians are informed that prescribed procedures have been performed or medications taken and receive answers to surveys which become part of a patient's medical history.

### BACKGROUND OF THE INVENTION

1. Field of the Invention

The present invention generally relates to patient healthcare monitoring by and communication with doctors and, more particularly, to a customizable system which can be used to aid patients with any medical condition in maintaining their health under the supervision of a doctor.

2. Background Description

Often, people, especially children and elderly persons, do not complete their necessary medical treatments as prescribed by their physician. Among the reasons for this is they forget to perform their treatment, the forget how to perform their treatment, or they refuse to perform their treatment. No one can benefit from medical treatment they are not receiving or that they do not receive properly. Patients need to be alerted to tasks that need to be completed and, when appropriate, patients need to be provided with short tutorials to instruct them as to how to properly perform procedures.

Weeks or months may pass between doctor visits, and patients often find it difficult to remember how they felt after taking a new medication, how long it took for a new medication to be effective, or to accurately report on problems with their health. Doctors need to be provided with timely and accurate information pertaining to medications that have been prescribed. In addition, there needs to be some record as to if and when the patient performs their treatment, and this record needs to be accessible by the patient's physician.

### SUMMARY OF THE INVENTION

It is therefore an object of the present invention to provide a system implemented on mobile devices that actively links doctors and patients in a collaborative fashion.

It is another object of the invention to provide a system which allows doctors to see how their patients are adhering to their treatment guidelines and, in addition, enables patients to be more responsible for their own treatment by putting more control in their own hands, with regular alerts and notifications when medication needs to be taken or other therapeutic action performed.

According to the invention, there is provided a comprehensive medical program, which reminds patients to perform medical treatments, demonstrates how to conduct treatments, and facilitates communication between doctors, patients and, in the case of children or the elderly, their parents or care givers. Patients are reminded through their handheld device to complete life-improving medical procedures while being walked through complicated procedures by means of pictures, graphic illustrations and instructions. These patients report their progress to their doctors through doctor-defined surveys. By reminding, instructing, tracking and reporting the success of patients following their doctor's orders, the invention provides a simple way to significantly improve the well being of those requiring medical care.

Patients are alerted when it is time to perform each medical procedure prescribed by their doctor. A single doctor can set these alerts, or a patient can receive alerts from multiple doctors. Doctors provide instructions and timetables for performing procedures and receive feedback about treatments using their tablet-based client. For example, a doctor may set an alert for a patient to take his or her medication at a certain time. Later, the patient is prompted to answer a survey about how he or she is feeling. For a child who does not respond to an alert, the system notifies their parent(s) (via e-mail or text message), who can take action to ensure the child complies with his or her medical treatment. Additional weekly reports are provided to the parent regarding their child's progress. Similarly, a care giver might be notified if an elderly patient does not respond to an alert.

Health surveys can be issued which allow doctors to quickly detect side effects and other issues related to a specific patient's care. If problems are detected, the doctor can then have the patient schedule an appointment to discuss treatment related issues.

### BRIEF DESCRIPTION OF THE DRAWINGS

The foregoing and other objects, aspects and advantages will be better understood from the following detailed description of a preferred embodiment of the invention with reference to the drawings, in which:

FIG. 1 is a block diagram of the application architecture of the preferred embodiment of the invention;

FIG. 2 is a screen print of the create patient input screen of the physician's tablet PC;

FIG. 3 is a screen print of the create instruction screen of the physician's tablet PC;

FIG. 4 is a screen print of an alarm generator screen of the physician's tablet PC;

FIG. 5 is a screen print of an alternative alarm generator screen of the physician's tablet PC;

FIG. 6 is an illustration showing a main alert screen of a patient client device;

FIG. 7 is an illustration showing a medical instructions screen of a patient client device;

FIG. 8 is an illustration showing a survey screen of a patient client device;

FIG. 9 is a block diagram illustrating an entity relationship of processes implemented by the invention;

FIG. 10 is a flow chart showing the procedure for login for the doctor on the doctor's tablet PC;

FIG. 11 is a flow chart showing the procedure for entering and editing patient data by the doctor;

FIG. 12 is a flow chart showing the procedure for creating or editing a treatment process;

FIG. 13 is a flow chart showing the procedure for setting up an alert schedule for a patient by the doctor;

FIG. 14 is a flow chart showing the procedure for reviewing patient compliance and treatment history by the doctor;

FIG. 15 is a flow chart showing the procedure for synchronizing the patient's handheld device;

FIGS. 16A and 16B, taken together, are a flow chart showing the procedure for notifying the patient to perform a treatment;

FIG. 17 is a flow chart showing the procedure for creating and editing a group by the doctor;

FIG. 18 is a flow chart showing the procedure for the doctor messaging system;

FIG. 19 is a flow chart showing the procedure for the patient messaging system;

FIG. 20 is a flow chart showing the procedure for saving patient data to a file; and

FIG. 21 is a flow chart showing the procedure for uploading patient data to the system database.

### DETAILED DESCRIPTION OF A PREFERRED EMBODIMENT OF THE INVENTION

The following scenarios will provide a basis for an appreciation of the invention. These scenarios are based on actual cases. In the following descriptions, the invention is referred to as “PocketDoc”, a trademark adopted for the invention by the inventors. While the following scenarios are based on actual cases, names have been altered for privacy.

Scenario 1—Dr. Samson, Susan, Ruth, Barbara

Dr. Samson is a urologist at Children's Hospital who has particular concerns about his patients. Several of his patients are supposed to be catheterizing themselves everyday; however, due to their high levels of urinary tract infections (UTIs), he believes they are not doing this properly. Dr. Samson uses the PocketDoc™ system as a new method of encouraging compliance from his patients. He creates patient profiles with a unique user interface designed to be similar to the clipboard system with which he is familiar. At first Dr. Samson was unsure about creating the tutorials for his patients, but after some practice he finds it easy to use to create tutorials to guide his patients through catheterization. After creating tutorials for patients, he can set up alerts for them and receive information regarding their progress via the messaging system included in the invention. Dr. Samson especially likes the ability to ask his patients questions about how they are feeling through the surveys. This allows him to pick up on early signs of a possible UTI.

Dr. Samson's favorite thing about using the PocketDoc™ system is the user interface. Designed to look like an office when opened, the interface is intuitive. Additionally, the use of a clipboard-like interface for creating patient alerts was something he was already familiar with so he did not have to adapt to something new to use the software. Dr. Samson is also pleased that the tablet is mobile and he can carry it around his office.

Susan is a 15 year old freshman in high school. She has been a patient of Dr. Samson's for five years. As part of her treatment she is supposed to catheterize herself several times a day. When Susan gets busy with school and other activities, she often forgets to catheterize herself or, by rushing, she fails to perform the procedure properly. When Dr. Samson introduced her to the PocketDoc™ system, she was apprehensive that it would function as an electrical leash. However, after using it for a week, she is sold. The alerts are quiet and, because of password protection, no one is able to find out about her treatment. Susan remembers to catheterize herself and makes fewer mistakes because she can follow the provided tutorial. She likes the program so much she convinced her diabetes doctor to use the PocketDoc™ system so that she can be reminded to check her blood sugar and communicate with him more easily when she is having problems.

Susan's favorite thing about the PocketDoc™ system is the ability to covert the alerting system which sounds like a telephone ringer and the password protected alerts, and she gets to carry a smart phone at school.

Susan's mother, Ruth used to worry about her daughter forgetting her medical procedures. However, now that Susan uses the invention, she does not have to worry all the time. If Susan misses an alert, a message is sent to Ruth informing her of the missed alert. If Susan is at school, Ruth calls the school nurse to correct things. If Susan is home, she simply reminds her to complete the given treatment.

Ruth's favorite thing about the PocketDoc™ system is that she and Susan fight less because she is not constantly nagging Susan about her treatment; rather, she only asks Susan about her treatments when she misses an alert.

Sometimes Susan misses an alert. Then Ruth calls Barbara, the school nurse. Barbara then calls Susan to her office to complete the treatment.

Barbara's favorite thing about the PocketDoc™ system is that it is a comprehensive treatment program which holds Susan accountable for her treatment yet provides a support system when Susan forgets. Since Susan has been using the PocketDoc™ system she is healthier and makes fewer visits to Barbara.

### Scenario 2—John, Mary, Foster Children, Family Pediatrician

John and Mary have their hands full. They have five foster children ranging from six months to ten years old. Each one has unique medical needs. John and Mary used to struggle to remember to give each child their medication. It was hard for them to keep up with each other and sometimes they both thought the other had treated a child and the treatment was forgotten. All that has changed since their family pediatrician introduced them to the PocketDoc™ system. She set up alerts for each of the children to help facilitate their treatment. Now John and Mary keep a PocketDoc™ client running on the kitchen table. Their PocketDoc™ client receives alerts for all their children. Now, no matter who is home at a given time when an alert goes off they take care of the treatment. Each alert has the name of the child and detailed instructions for what medications to give.

Their favorite thing about the PocketDoc™ system is a history kept for each child when they give feedback on questionnaires. This expedites doctor's visits as the doctor can preview information about the children. Also in case of a trip to the emergency room, each child's individual history and list of medications is easily accessible.

### Scenario 3—Rose (80), Manny (85), their Doctor

Rose is an 80-year-old woman who has been married to Manny, 85 for 50 years. Now Manny's health is failing and Rose is struggling to take care of him. There is Alzheimer medication, blood pressure medication, calcium pills and nebulizer treatments for emphysema. Rose does not want to put Manny in a home but she is concerned that she will forget something about Manny's treatment. Rose was very relieved when their doctor recommended she try the PocketDoc™ system. He set up alerts so that Rose is audibly alerted when Manny needs medication or a medical procedure. The detailed instructions include everything from how much of each medicine to administer and to how to set up and clean the nebulizer. When Rose misses an alert, one of the nurses from her doctor's office or sometimes even their own doctor calls her to remind her.

Rose's favorite thing about the PocketDoc™ system is that she does not have to worry as much about forgetting to treat Manny or having to put him in a home.

The preferred embodiment of the PocketDoc™ system on which the above scenarios were based was implemented with commercially available hardware and software tools, which will be mentioned as part of the following description. However, it will be understood by those skilled in the art that other and different hardware and software tools can be used in the practice of the invention.

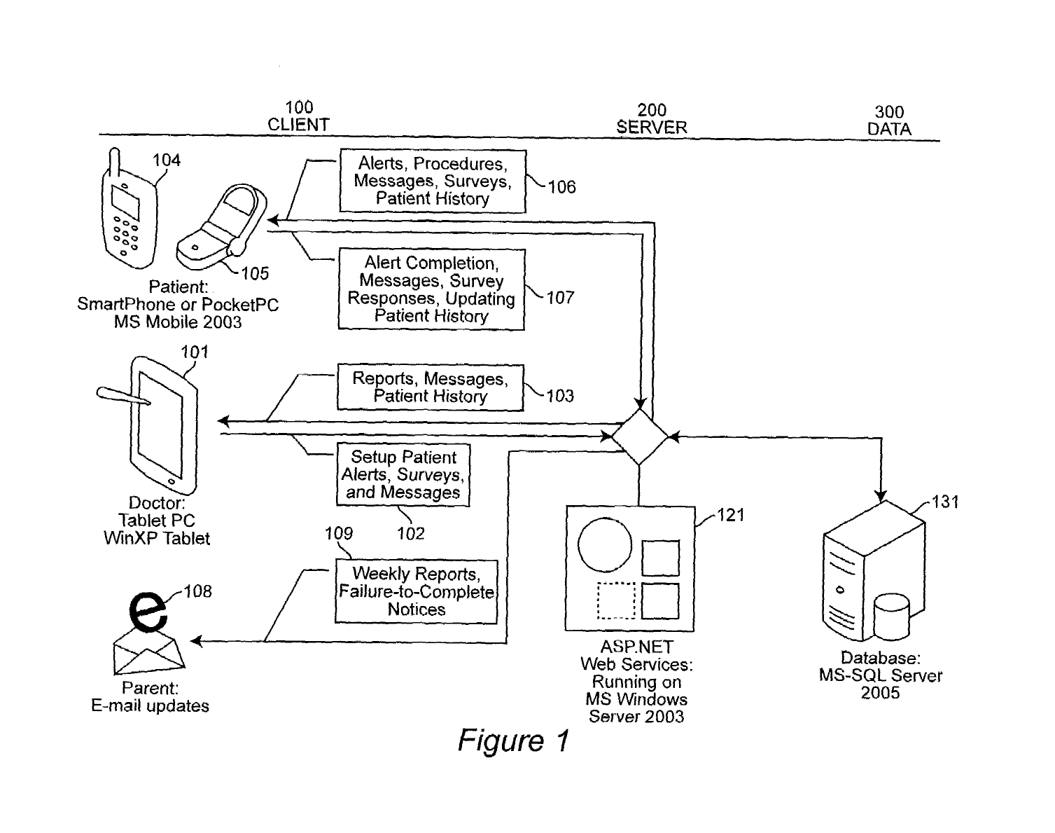

Referring now to the drawings, and more particularly to FIG. 1, there is shown the PocketDoc™ architecture diagram according to the preferred embodiment of the invention. This architecture is divided into three groups: the client group 100, the server group 120, and the data group 130. Beginning first with the doctor client 101 in the client group 100, this is preferably a tablet PC (personal computer) running the MS Windows XP Tablet PC edition operating system from Microsoft Corporation. While other mobile or portable computers could be used for the doctor client 101, the tablet PC was selected to provide the physician with a familiar user interface since it is similar to a clipboard. Using the doctor client 101, the physician can setup patient alerts and generate surveys and messages for each patient using the system, as generally indicated at 102. These are transmitted to the server 121, in the server group 120, which, in turn, stores and accesses this information on the database 131, in the data group. In the preferred embodiment, the server 121 runs MS Windows Server 2003 from Microsoft Corporation as the server or network operating system, which hosts Web services. Also, in the preferred embodiment, the data base runs MS SQL Server 2005 from Microsoft Corporation which hosts the PocketDoc™ database. The doctor client 101 receives from the server 121 various reports, messages and patient history from the server 121, as generally indicated at 103.

Considering next the patient client, the patient client can be implemented on a variety of mobile or portable devices, including a smart phone 104, a pocket PC 105, as shown, or other devices such as a PDA (personal digital assistant). A smart phone is defined as any electronic handheld device that integrates the functionality of a mobile phone with a PDA or other information appliance. A pocket PC is defined as a handheld-sized PC that runs, for example, Windows Mobile (formerly known a Windows CE) from Microsoft Corporation. Other operating systems for both the smart phone and the pocket PC, such as the Palm OS developed by the PalmSource, may be used. The specific device chosen can be different from patient-to-patient according to the individual patient's personal preferences. In the preferred embodiment, the patient client MS Mobile 2003 from Microsoft Corporation as the operating system. The patient client receives, via the server 201, alerts, procedures, messages, surveys, and patient history, as determined by the patient's physician, and as generally indicated at 106. The patient client also transmits to the server 201 information including alert completion, messages, survey responses, and updated patient history, as generally indicated at 107.

In the illustration of FIG. 1, it will be understood that there are multiple doctor clients and multiple patient clients, and as indicated by the above scenarios, multiple patients may be under the care of multiple doctors. Moreover, while there is illustrated but one server 121 and one database 131, those skilled in the art will recognize that multiple servers and multiple databases may be used in a practical embodiment. And while the communications are indicated as being direct between the doctor clients, patient clients, server and database, in a practical embodiment a variety of communication links may be implemented. For example, for short distances the Bluetooth wireless standard may be used for communications between clients and a local server. Clients may also communicate via the Internet with the server 121 as may also the server 121 communicate with the database 131. And as is well known in the art, communication via the Internet may involve a variety of modalities, including copper wire, coaxial cable, fiber optic cable, satellite, etc. Therefore, the description provided and illustrated with respect to FIG. 1 is intended to be merely exemplary of the overall architecture of the system, and those skilled in the art will recognize that specific implementations will vary from application to application.

From this general description, it will be appreciated that the PocketDoc™ system according to the invention provides a unique and highly customizable way to facilitate physician/patient communications. In addition, and especially for children, the PocketDoc™ system can provide e-mail updates 108 to parents. These can be in the form of weekly reports, failure-to-complete notices, and the like, as generally indicated at 109. Of course, such e-mail communications can be provided to spouses, guardians and other care providers for patients such as Manny described in scenario 3, above.

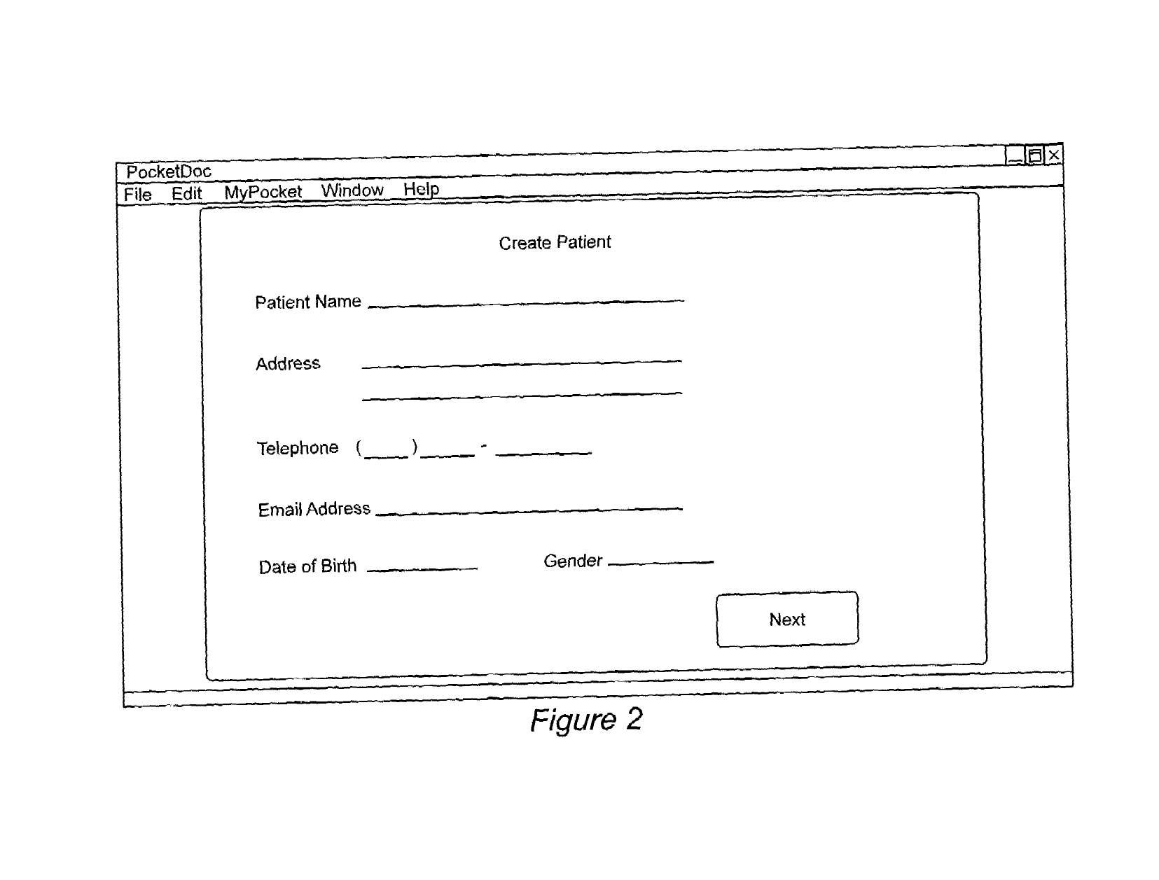

FIG. 2 is a screen print of an example of a graphic user interface on the doctor client 101 using a Tablet PC. This is the initial patient data entry screen which prompts for such information as the patient's name, address, telephone number, e-mail address, date of birth and gender. The physician can use a stylus (typically supplied with the Tablet PC) to enter this information, in the same manner as writing on a form on a clipboard.

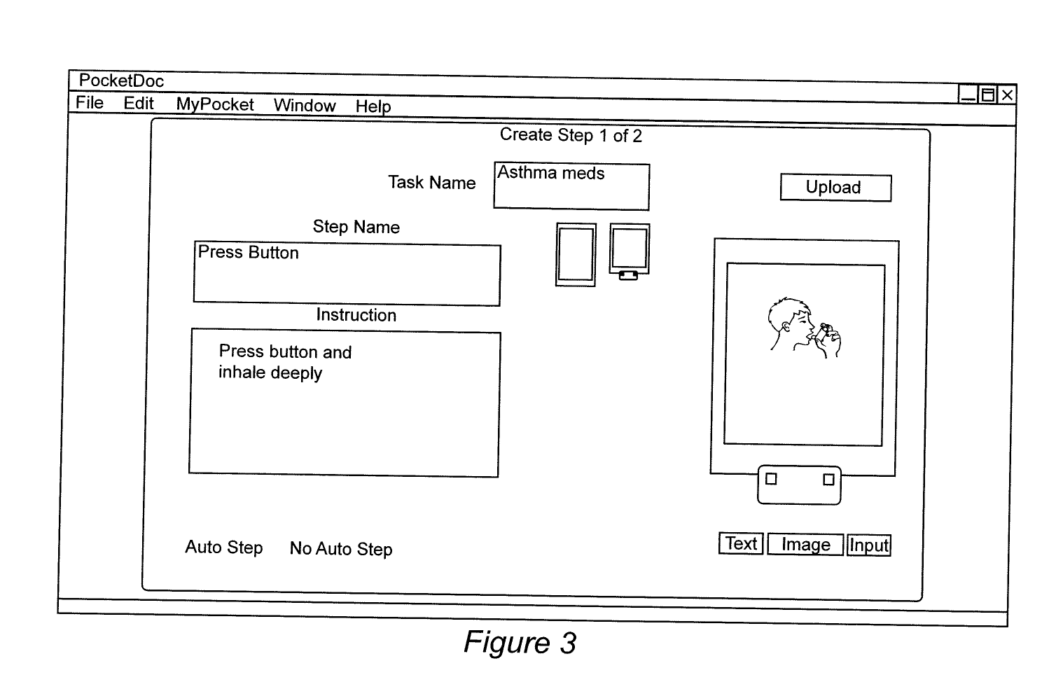

FIG. 3 is a screen print of an example of a graphic user interface on the doctor client 101 using a Tablet PC to enter instructions or a tutorial for performing a particular medical procedure, in this case the use of nebulizer. The task name is identified as “Asthma meds” and the step name is “Press Button”. The physician has used the stylus to write as the instruction “Press button and inhale deeply”. On the right hand side of the tablet display is an illustration of the patient client device showing the graphic illustration of the procedure. In each procedure initiated on the doctor client 101, there is an illustration of the patient client so that the physician will be able to confirm that the graphics or images generated on the patient client are what the physician intends. Below the patient client illustration are a series of buttons labeled “Text”, “Image” and “Input”. In the illustrated example, the button “Image” has been pressed. The physician can scroll through a series of images to choose the image he or she wants to accompany the text instruction. It is also possible for the physician to sketch a diagram if there is not a predefined image he or she considers suitable for accompanying the instruction.

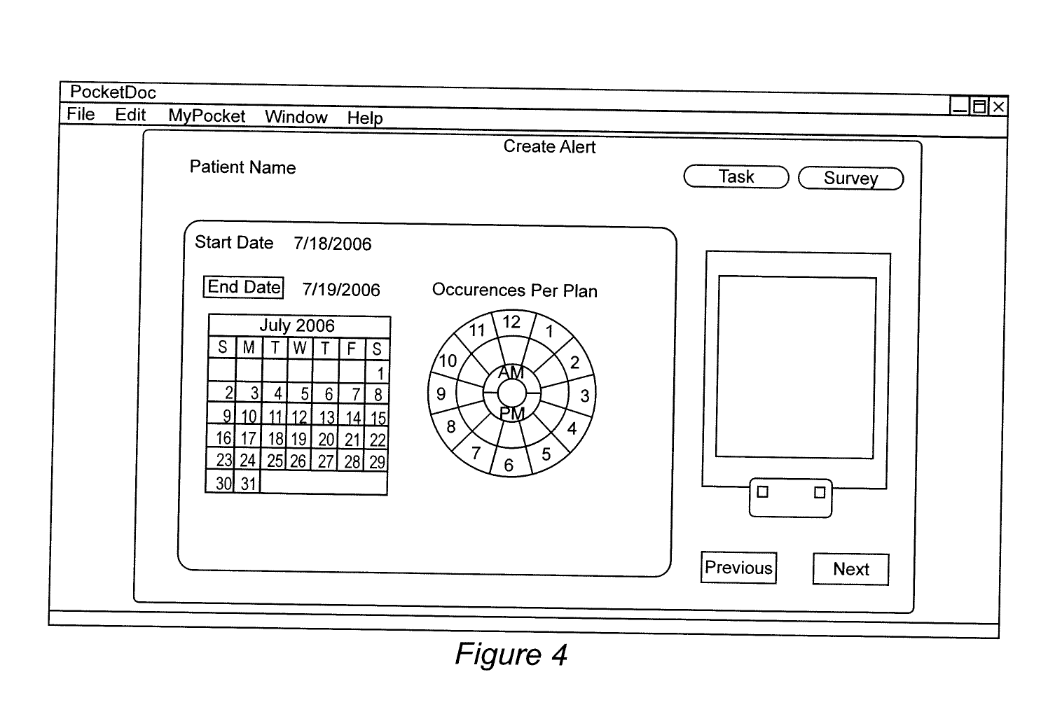

FIG. 4 is a screen print of an example of a graphic user interface on the doctor client 101 using a Tablet PC for creating an alert for the patient. In this screen, the patient name is first entered in the upper left corner by pressing the down arrow key and selecting the patient name from a list of previously entered patient names. Next, the start date is selected by pressing the start date button and then selecting a day on the calendar below. This is followed by pressing the end date button and selecting a date on the calendar. Finally, the occurrences per day is selected using the circular graphic in the center of the screen. This graphic is first divided into twelve segments in the outer two rings. The outermost ring segments are labeled from one to twelve, corresponding to hours, and the adjacent ring segments are labeled from 0 to 55 in increments of five, corresponding to minutes. The next ring is divided into two segments, corresponding to AM and PM time periods, while the center circle is the “Enter” button. To use this graphic, the physician enters the time of day, say for example 7:30 AM, by pressing the 7 in the outermost ring, then the 30 in the next adjacent ring, then the AM segment, and finally the Enter button. If procedures or medications are to be repeated multiple times a day, the process is repeated for each time.

Again, on the right hand side of the screen is a graphic representation of the patient client. Above this graphic representation are two buttons, one labeled “Task” and the other labeled “Survey”. By pressing the “Task” button, the physician can enter the nature of the task to be performed by the patient at the time of the alert. By pressing the “Survey” button, the physician can generate a series of questions to be posed to the patient at some predetermined time after the patient has performed the procedure or taken the medication prescribed by the task.

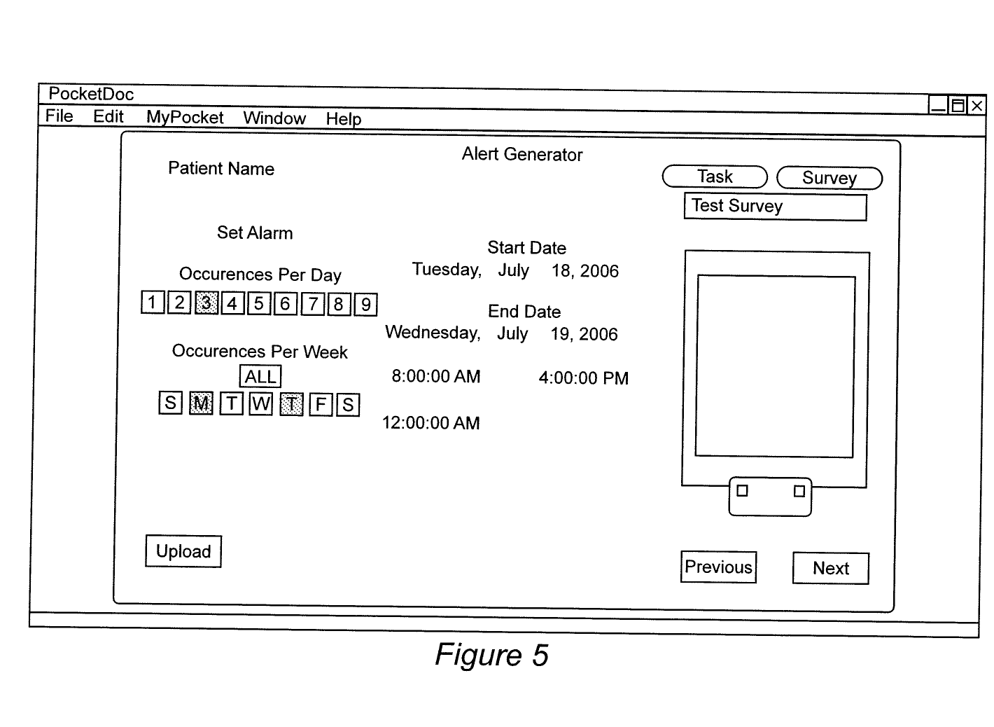

FIG. 5 is an alternative screen print for the alarm generator. Again, in the upper left corner is a place to enter the patient's name. This done in the same manner by pressing the down arrow and selecting the patient's name. Next, the start date and end dates are entered by pressing the respective down arrows and selecting a date on a calendar which is displayed in response to pressing the down arrows. Next, the physician selects the number of occurrences per day. In the example shown, the “3” button has been selected. Depending on which button is selected, a number of windows for the time of day is opened. Since the “3” button has been selected in this example, three windows have been opened. The times are entered by selecting the up and down arrows for each of the windows. Finally, the physician selects the occurrences per week. In the example shown, the days of Monday and Thursday have been selected.

It will be observed in FIG. 5 that a survey is in the process of being generated in the illustration of the patient client. Notice that the form of the survey is in the form of multiple choices, here labeled “a”, “b” and “c”.

In summary, the application components of the PocketDoc™ client for physicians
Run on a Tablet PC, chosen because it mimics the pen-based environment doctors use
Feature an innovative user interface
Allow for scheduling of alerts
Provide a special tool for creating tutorials
Allow for the creation of surveys
Send and receive messages
Provide physicians with survey responses
Calls Web Services to facilitate communication with a central PocketDoc™ server

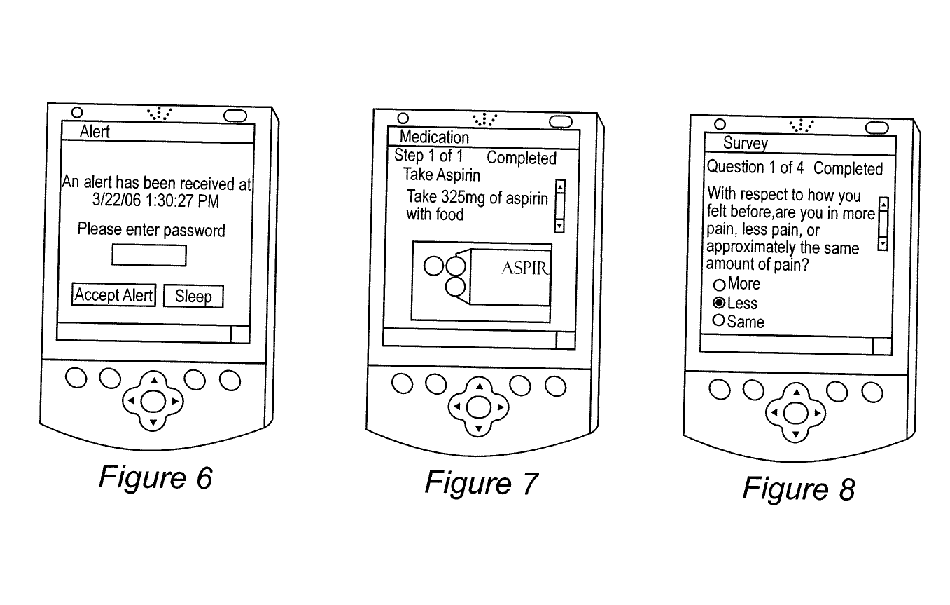

Turning next to the patient client, FIG. 6 is a screen print showing that an alert has been received. This screen shows the time the alert was received and prompts the user to enter his or her password. After entering the password, the user either presses the “Accept Alert” button or the “Sleep” button. The latter might be pressed if the patient were not in a convenient location, such as public transportation, for performing the prescribed medical procedure or taking the prescribed medication. If the “Sleep” button is pressed, the alert would be repeated at a predetermined time interval which could be set in system preferences at the server 121 and/or the patient client. By pressing the “Accept Alert” button, the patient indicates that he or she is ready to perform the medical procedure or take the medication.

FIG. 7 is a screen print of the patient client display after the “Accept Alert” button has been pressed. This display provides the patient with the medical instructions generated by the physician as described above. In the simple example illustrated, the top half of the display is the text of the medical instruction, while the bottom half of the display is either a graphic or video illustration of the process of described in the text above. More specifically, the text instructs the patient to take aspirin with food, and the illustration shows an aspirin dispenser. When the patient has completed the process or taken the medication, the patient presses the “Completed” button in the upper right corner of the screen, indicating compliance with the physician's instructions. This is communicated back to the server 121 which records it in the database 131.

One of the important features of the present invention is the ability to get contemporary feedback from the patient. As previously described, the physician as part of generating alerts can also generate surveys which are presented to the patient. An example of one of these is shown in FIG. 8. A typical survey will contain a series of questions, typically on the order of one to six questions. The survey may be presented to the patient immediately after the patient has indicated that he or she has completed a medical procedure, or the survey may be delayed for a period of time to allow a medication to have an effect on the patient. In the example shown, the patient is given three choices to select concerning his or her level of pain. These are “More”, “Less” and “Same”. After making a selection, the next question is displayed by pressing the down arrow to the right of the question. The patient can return to a previous question by pressing the up arrow and, if desired, change a selection. Once all the questions of the survey have been answered, the completed button in the upper right corner is pressed.

In summary, the PocketDoc™ client for patients
Runs on a mobile device (Pocket PC or Smart Phone)
Alerts patients to a scheduled medical procedure
Features password protected alerts
the PocketDoc server when a patient misses an alert so a parent or third party can be informed
Displays surveys and gathers and saves patient responses
Sends and receives messages from their physician
Automatically synchronizes with the central PocketDoc™ server when connected to the Internet
Calls Web Services to facilitate communication with a central PocketDoc server

The PocketDoc™ server
Hosts the PocketDoc™ database
Protects patient privacy by storing patient information in an encrypted format
Utilizes encryption when sending and receiving messages
Hosts Web Services used by the doctor and patient client applications
Generates generic messages to parents when a child has missed and alert

In addition to the software already mentioned, various programming languages and tools were used in the implementation of the PocketDoc™ system. These include C# (C sharp, an object oriented programming language) and XML (extensible Markup Language), but again, those skilled in the art will recognize that other programming languages and tools can be used in the practice of the invention.

The following is an example of the XML code used to generate how steps in the alert are displayed to the patient in the example of FIG. 7.

```
// In this method LINQ is used to query an existing XMLDocument and
// return a List of steps for that alert.
private List<XElement> getSteps( )
{
  IEnumerable<XElement> stepsInAlert =
    from
      c in patientAlerts.Element(“alerts”) .Elements(“alert”)
        .Elements(“step”)
      where
        c.Parent.Element(“alert_id”)
        .Value == “1”
      select c;
  List<XElement>steps = new List<XElement>O;
  foreach(XElement step in stepsInAlert)
    steps.Add(step);
  return (steps);
}
// This section of code is used each time a new step is to be displayed
// to the user.
private void displayNextStep( )
{
  //if there are more steps set the form properties
  if (stepIndex < numSteps)
  {
    this.StepProgressLabel.Text = (“Step ” + (stepIndex+1) + “ of ”+
numSteps);
    this.StepName.Text = “” + steps[stepIndex].Element(“step_name”)
        .Value;
    this. InstructionBox.Text=“”+
    steps[stepIndex].Element(“instruction”)
        .Value;
    if(“”+steps[stepIndex].Element(“input”).Value == “true”)
    {
        this.InputBox.Visible = true;
    }
    else
    {
        this.InputBox.Visible = false;
    }
    if(“”+steps[stepIndex].Element(“picture”).Value == “none”)
    {
        this.Image.Visible = false;
    }
    else
    {
        pictureURL = steps[stepIndex].Element(“picture”).Value;
        this.Image.Visible = true;
        loadImage( );
        }
    }
    //no more steps, so exit
    else
    {
        this.Close( );
    }
    stepIndex++;
}
PocketPatient.xml
<alerts>
  <alert>
    <alert_id>|</alert_id>
    <time>9:00</time>
    <name>Medication</name>
    <task>true</task>
    <step>
      <step_number>|</step_number>
      <step_name>Take Asprin</step_name>
      <instruction>Take 325 milligrams of aspirin with
          food.</instruction>
        <input>false</input>
        <picture>c:\PocketDoc\Images\aspirin.gif</picture>
    </step>
  </alert>
  <alert>
    <alert_id>2</alert_id>
    <time>10:30</time> <name>Catheterization</name>
    <task>true</task>
    <step>
      <step_number>|</step_number>
      <step_name>Take Medicine</step_name>
      <instruction>Take x milligrams of aspirin</instruction>
      <input>false</input>
      <picture>c:\PocketDoc\Images\aspirin.gif</picture>
    </step>
    <step>
      <step_number>2</step_number>
      <step_name>do something</step_name>
      <instruction>what to do</instruction>
      <input>false</input>
      <picture>c:\PocketDoc\Images\aspirin.gif</picture>
    </step>
  </alert>
</alerts>
```

Form the foregoing, it will be appreciated that the PocketDoc™ system is a comprehensive medical program, which reminds patients to perform medical treatments, demonstrates how to conduct treatments, and facilitates communication between doctors, patients and, in the case of children or elderly persons, their parents or care givers. Patients are reminded through their handheld client device to complete life-improving medical procedures while being walked through complicated procedures by means of pictures and instructions. These patients report their progress to their physicians through doctor-defined surveys. By reminding, instructing, tracking an reporting the success of patients following their physician's orders, the PocketDoc™ system provides a simple way to significantly improve the well being of those requiring medical care,

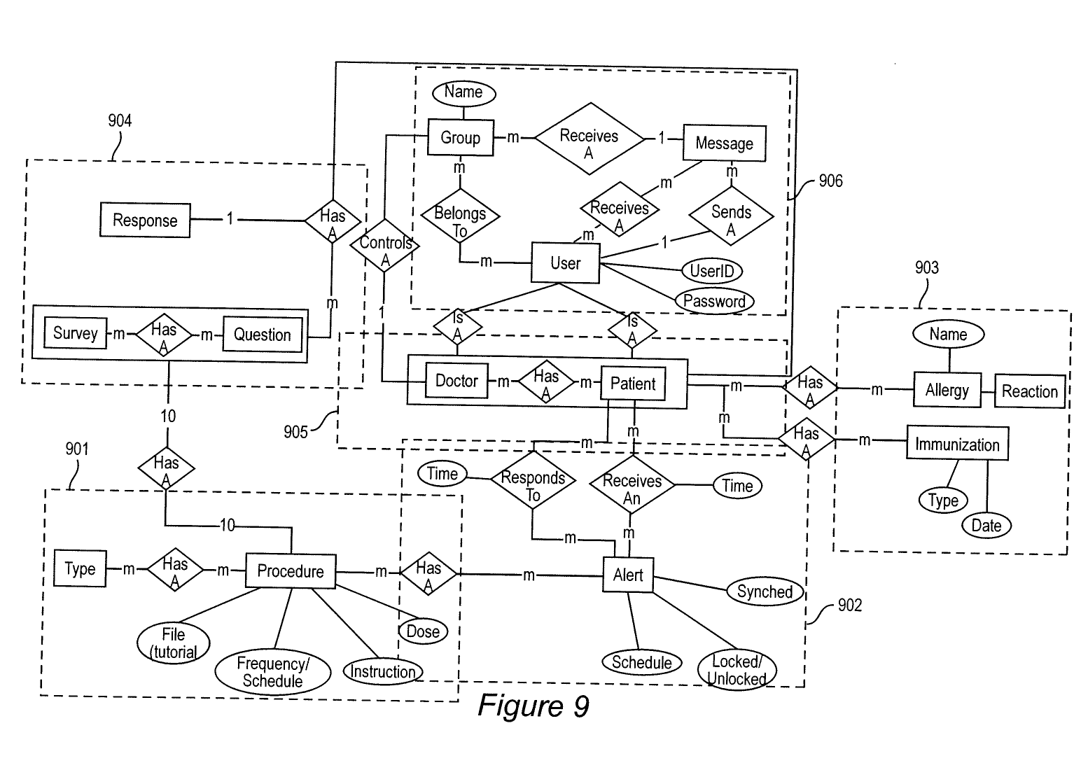

FIG. 9 provides a summary of the entity relationships in the PocketDoc™ system. In the procedures entity 901, an individual task such as taking a prescription (e.g., taking 400 mg Motrin with plenty of water), performing a medical procedure such as taking glucose levels, or personal reminders such as preparing food prior to medication is represented. The alerts entity 902 includes a schedule of procedures (part of the procedures entity 901) and what times to perform them. The emergency medical information entity 903 includes such information as medical allergies, food allergies, current immunizations, etc. The survey and responses entity 904 is a specialized procedure (i.e., part of the procedures entity 901) that enables the doctor to set up questions that the user needs to answer. This entity is used in conjunction with the alerts entity 902 to set up predefined questions that are asked at specified times and intervals. The doctor/patient information entity 905 is used to link all information pertaining to alerts, surveys, and the like. Finally, the groups/message entity 906 allows messages to pass between doctors and patients. This entity relationship diagram provides a map of the software implemented in the PocketDoc™ system and described in more detail with reference to the following flowcharts.

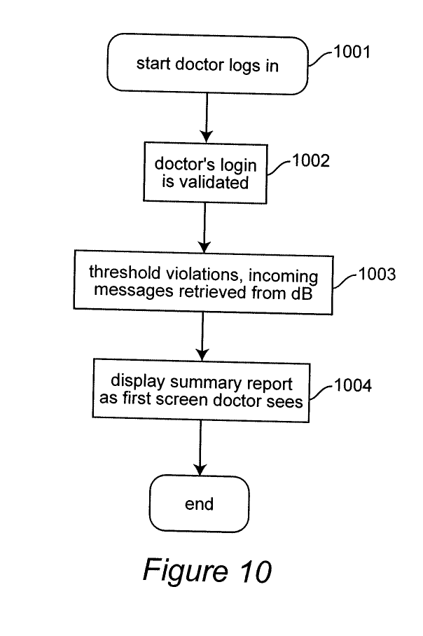

FIG. 10 is the flowchart illustrating the doctor login process. The process begins in function block 1001 where a login screen is displayed on the physician's tablet PC. This login screen includes, for example, fields for entering the physician's name and perhaps a personal identification number (PIN). This might be accomplished manually or, in the alternative, the login could be accomplished with a USB (universal serial bus) “key”, such a the Griffin Technologies SecuriKey USB device, which is inserted into a USB port on the tablet PC. Next, in function block 1002, the login is validated, and once validated, the tablet PC retrieves threshold violations and incoming messages from the database in function block 1003. Then, in function block 1004, a summary report is displayed as the first screen the doctor sees after login.

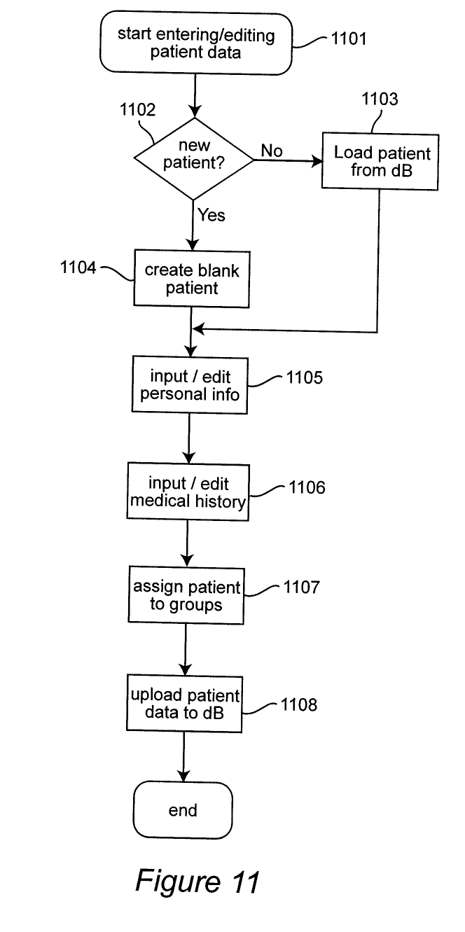

FIG. 11 is the flowchart for the process of entering and editing patient data on the doctor's tablet PC. The process begins in function block 1101 by selecting the entering/editing patient data button on the user interface. The first determination to be made in the decision block 1102 is whether the patient is a new patient. This can be as a result of asking the doctor to select between a new or current patient button, but preferably is an automated process in which the doctor simply enters the patient's name and the system makes the determination. So, if the patient is a current patient, as determined by a search of the database, the patient information is automatically loaded from the database in function block 1103. If the patient is not found in the database, the screen shown in FIG. 2 is displayed in function block 1104 for the doctor to enter patient information. In function block 1105, the personal information input or edited by the doctor is received by the system, and in function block 1106, the medical history input or edited by the doctor is received by the system. The doctor can also assign the patient to one or more groups, and this information is received by the system in function block 1107. Finally, the data input by the doctor is uploaded to the database in function block 1108.

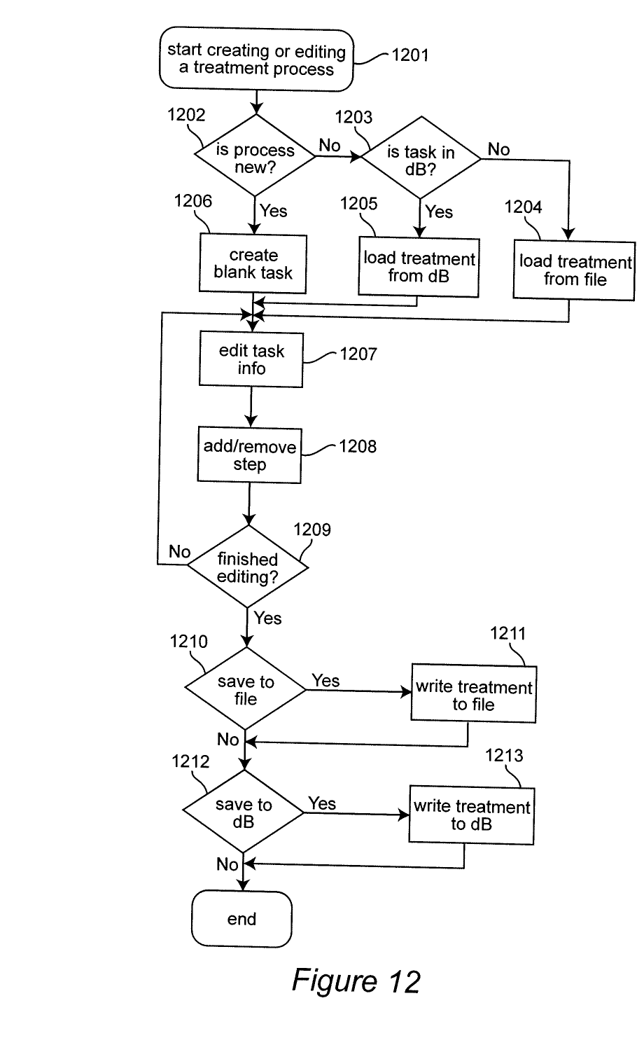

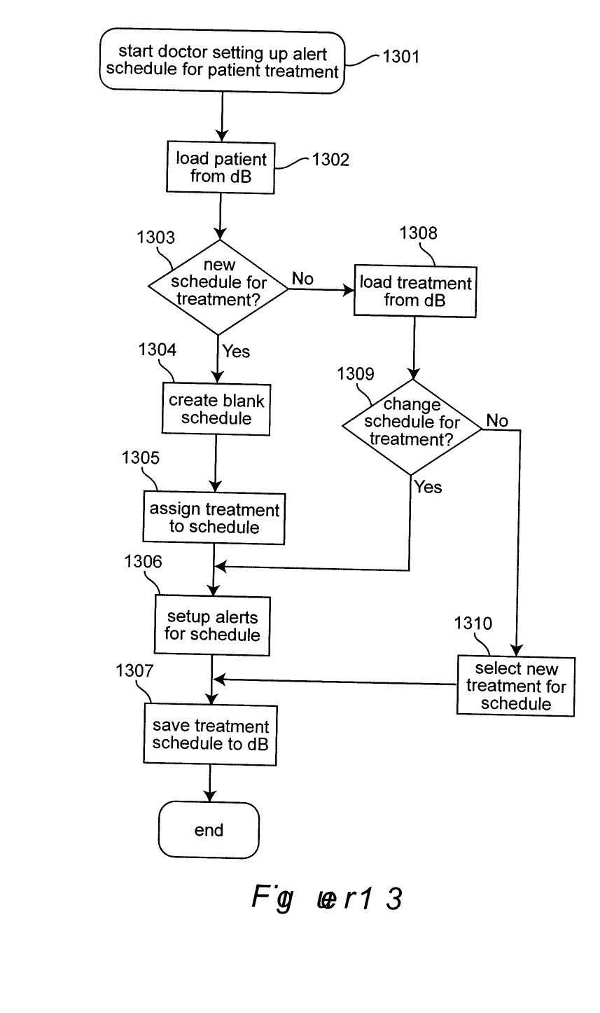

FIG. 12 is the flowchart for the process of creating or editing a treatment process for a patient by the doctor. The process begins in function block 1201 by selecting the create/edit treatment button on the user interface. In decision block 1202, a determination is made as to whether the process is new. This can be done by prompting the doctor to make an appropriate selection. If the process is not new, a determination is made in a decision block 1203 as to whether the task is in the database.

This can be automated by a search of the database for the task entered by the doctor. If the task is in the database, the treatment is loaded in function block 1204; otherwise, the treatment is loaded from a file input to the tablet PC in function block 1205. In the case that the process is new, a blank task screen is displayed in function block 1206. Once the task information is input or loaded, it can be edited. The edited task information is received by the system in function block 1207. If a step is added or removed, that information is received by the system in function block 1208. A determination is made in decision block 1209 as to whether editing is completed. This may be determined by the doctor selecting a appropriate button on the user interface of the tablet PC. If not finished editing, a return is made to function block 1207; otherwise, a determination is made in decision block 1210 as to whether the information should be saved to a file. Again, this may be determined by the doctor selecting an appropriate button on the user interface. If the information is to be saved to a file, the treatment is written to the file in function block 1211; if not, a determination is made in decision block 1212 as to whether the information is to be saved to the database. Once again, this may be determined by the doctor selecting an appropriate button on the user interface. If the information is to be saved to the database, the treatment is written to the database in function block 1213 before the process ends.

FIG. 13 is a flowchart of the process of the doctor setting up an alert schedule for a patient treatment. The process begins in function block 1301 by selecting the alert schedule button on the user interface. The doctor enters the patients name, and in function block 1302, the patient information is loaded from the database. The doctor is prompted in decision block 1303 to indicate whether this is a new schedule for treatment. If it is, a create blank schedule screen is displayed in function block 1304. The treatment information entered by the doctor is received by the system in function block 1305. Then the alerts for the schedule entered by the doctor are received by the system in function block 1306, and the treatment schedule is saved to the database in function block 1307. Returning to decision block 1303, if it is not a new schedule for treatment, the treatment is loaded from the database in function block 1308. Once the treatment is loaded, the doctor is prompted in decision block 1309 to indicate whether there is a change schedule for the treatment. If so, the process goes to function block 1306 where alerts for the schedule are input; otherwise, a new treatment for scheduling is selected in function block 1310, and the process goes to function block 1307.

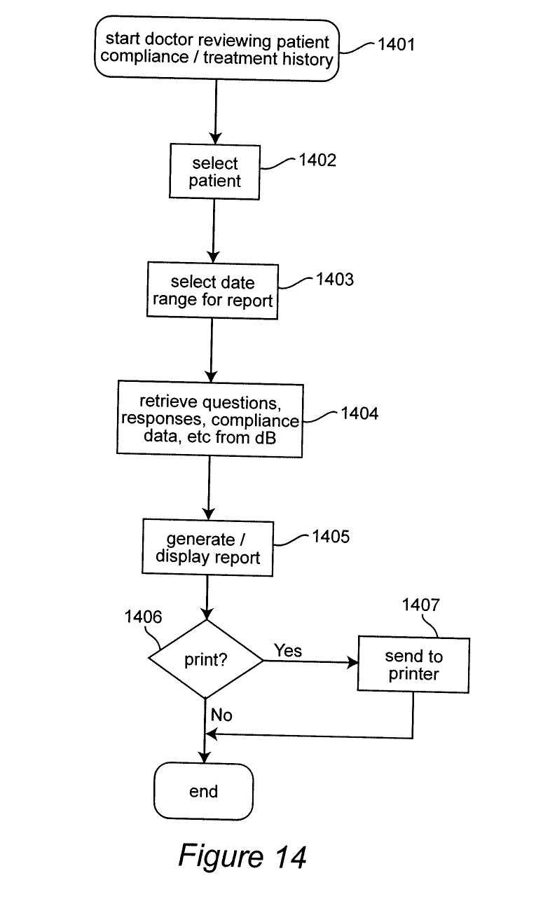

FIG. 14 is a flowchart of the process of the doctor reviewing patient compliance and treatment history. The process begins in function block 1401 by selecting the patient compliance/treatment history button on the user interface. The doctor is prompted to select a patient in function block 1402. This can be done by the doctor writing in the patient's name or displaying a list of patient names from which the doctor may select a patient. After selecting a patient, the doctor is prompted to select a date range for the report in function block 1403. In response to the a date range being selected, questions, responses, compliance data, and the like are retrieved from the database in function block 1404. The retrieved information is formatted and a report is generated and displayed in function block 1405. The doctor is prompted in decision block 1406 as to whether the report is to be printed. If so, the report is sent to the printer in function block 1406; otherwise, the process ends without printing a report.

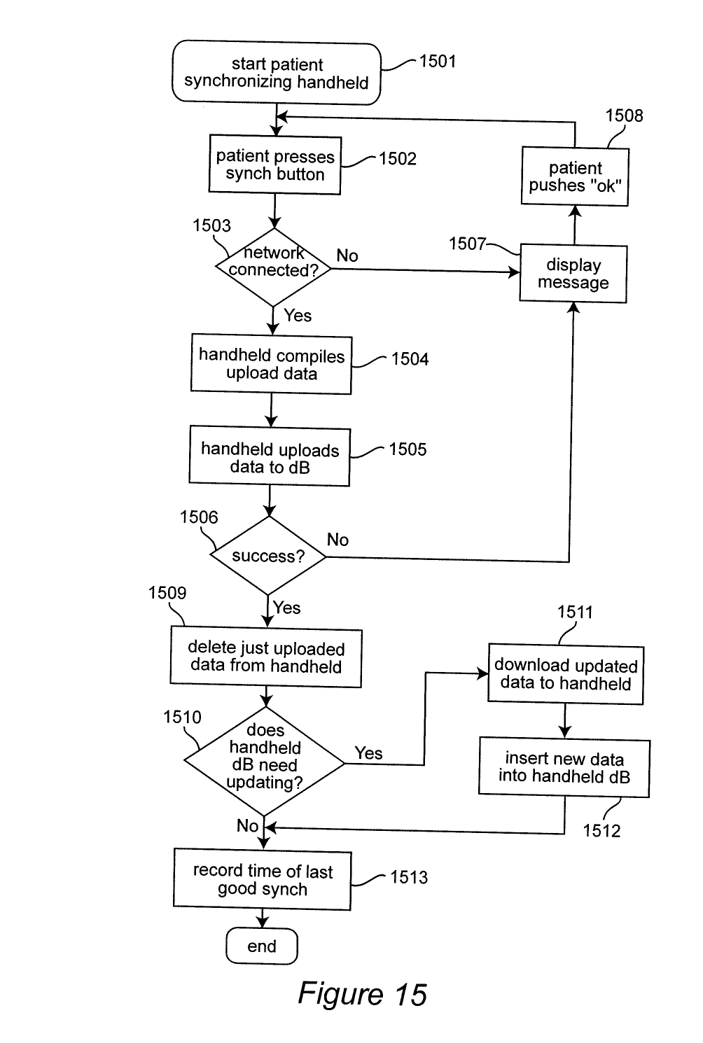

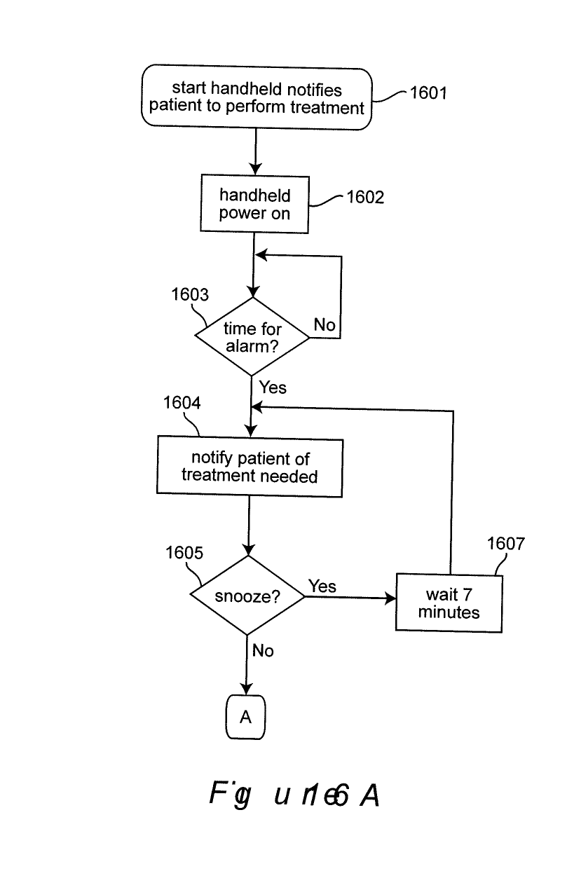

FIG. 15 is a flowchart of the process for synchronizing the patient handheld device (e.g., smart phone, pocket PC, PDA). The process begins in function block 1501 by selecting the PocketDoc™ function on their handheld device. Then, in response to the patient pressing the sync button in function block 1502, the device makes a determination in decision block 1503 as to whether the device is currently connected to a network, typically the Internet. If so, the handheld device compiles data for uploading in function block 1504, and the compiled data is uploaded to the system database in function block 1505. A determination is made in decision block 1506 as to whether the data has been successfully uploaded and, if so, the uploaded data is deleted from the handheld device in function block 1507. If the determinations made in either of the decision blocks 1503 or 1506 is negative, then a message to that effect is displayed in function block 1508, and the patient is prompted to press the “OK” button in function block 1509. Upon detecting that the “OK” button is pushed, the process returns to function block 1502, where the patient is prompted to press again the sync button. Returning to function block 1507, once the data is deleted, a determination is made in decision block 1510 as to whether the handheld device database needs updating. If so, updated data is downloaded to the handheld device in function block 1511, and the new data is inserted into the handheld device database in function block 1512. At this point, the time of the last good synch is recorded in function block 1513 before the process ends.

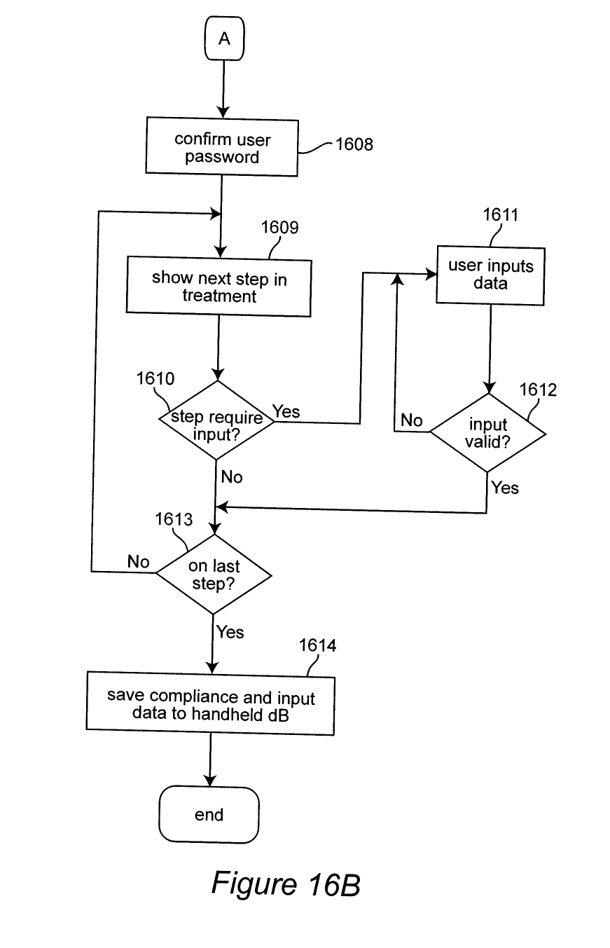

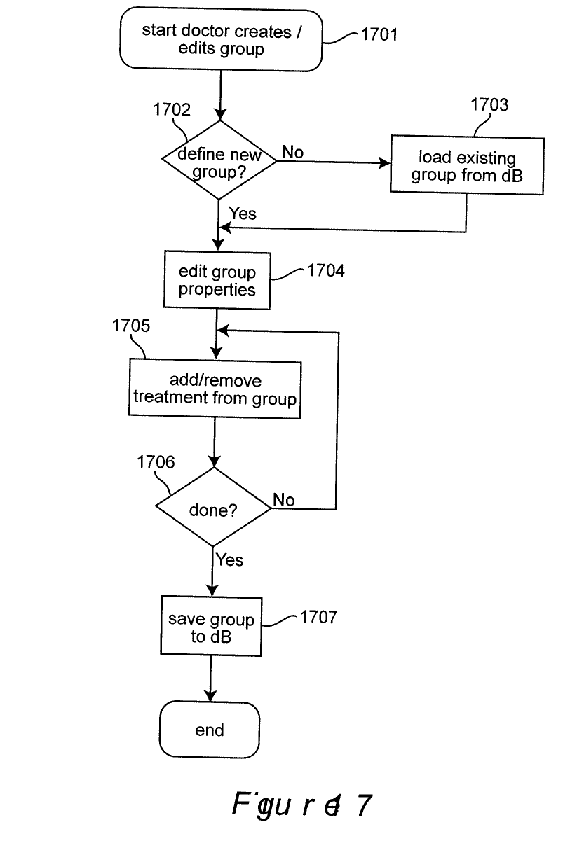

FIGS. 16A and 16B, taken together, are a flowchart of the process of the patient notification process by the handheld device. The process begins in function block 1601 upon power on in function block 1602. The process continuously monitors alarm times and determines in decision block 1603 when an alarm should be generated. The alarm which is generated in function block 1604 by the handheld device may be audible, vibratory or other sensory alarm as may be built into the handheld device. Once the alarm is generated, the patient has the option of delaying response to the alarm by pressing what amounts to a “snooze” button, as detected in decision block 1605. If this button is pressed, the handheld waits for some predetermined period of time, say seven minutes, before again notifying the patient in function block 1604. The user is next prompted to confirm his or her user password in function block 1606. As mentioned, this is a feature which provides the patient with privacy. Once the user password has been entered and confirmed, the handheld displays by text, graphic, picture or a combination thereof a first or next step in the treatment in function block 1607. A determination is made in decision block 1608 as to whether the displayed step requires user input. If so, the user input data is received in function block 1609 and a determination is made in decision block 1610 as to whether the user input is valid. If not, the user will be prompted to again input data in function block 1609. Then, in decision block 1611, a determination is made as to whether the last step of the treatment has been displayed and, if not, the process returns to function block 1607 where the next step in the treatment is displayed. Once all the steps have been displayed, compliance information and patient input data is saved to the handheld database in function block 1612 before the process ends.

FIG. 17 shows the flowchart for the process of the doctor creating or editing a group. The process begins in function block 1701 by selecting the create/edit group function on the user interface of the doctor tablet PC. The doctor is prompted in decision block 1702 to indicate whether a new group is to be defined. If not, an existing group is selected and loaded from the system database in function block 1703. A screen is displayed in function block 1704 for editing group properties. Information that treatments that are added or removed from the group are received in function block 1705. A determination is made in decision block 1706 as to whether all edits are completed. This may be done by receiving an input from an appropriate button on the user interface pressed by the doctor. When the edits are complete, the group is saved to the system database in function block 1707 before the process ends.

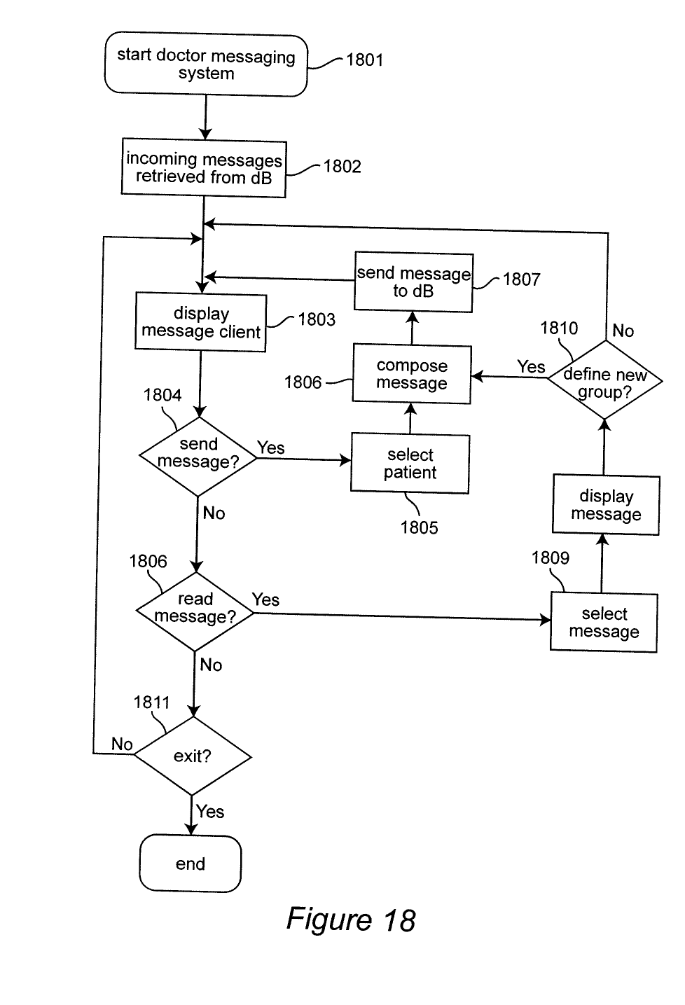

FIG. 18 shows the flowchart for the process implemented for the doctor messaging system. The process begins in function block 1801 by selecting the messaging function on the user interface of the doctor tablet PC. Incoming messages are retrieved from the system database in function block 1802. The message client is displayed on the doctor's tablet PC in function block 1803. The doctor can send a message or read a message. If the doctor chooses to send a message, as determined in decision block 1804, the doctor is prompted to select a patient in function block 1805. Once a patient is selected, the doctor is prompted to compose a message in function block 1806. The composed message is then sent to the system database in function block 1807, from which it is sent to the patient, as generally indicated in the system diagram of FIG. 1. If the doctor chooses to read a message, as determined in decision block 1808, the doctor is prompted to select a message in function block 1809. The selected message is displayed in function block 1810. A determination is made in decision block 1811 as to whether the doctor wishes to reply. If the doctor wishes to reply, the process goes to function block 1806 where the doctor composes a reply. Finally, the doctor can choose to exit the messaging system as detected by decision block 1812.

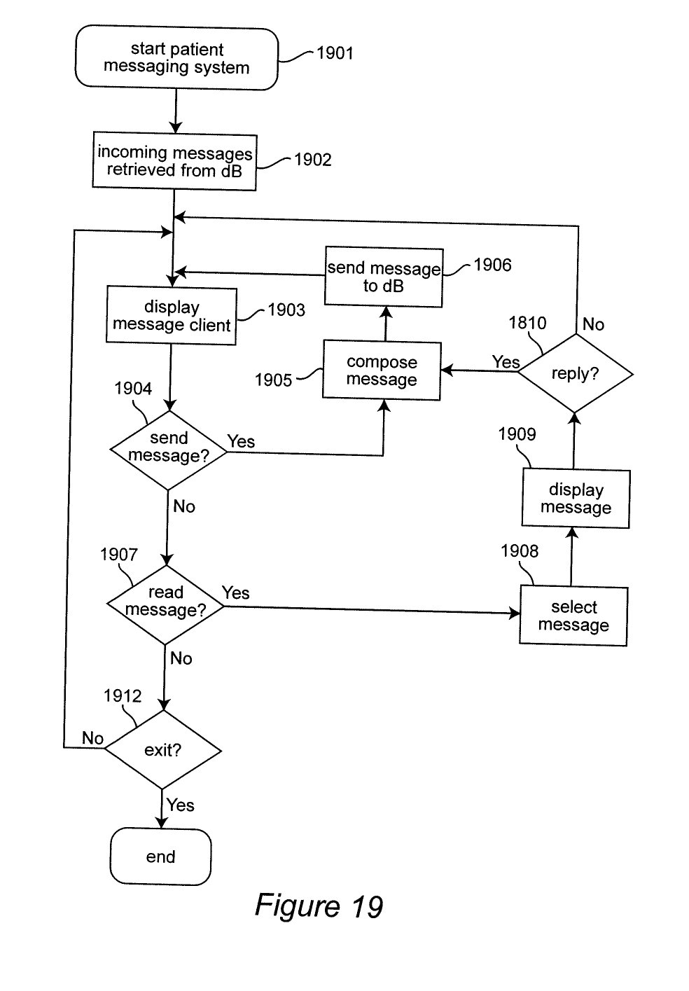

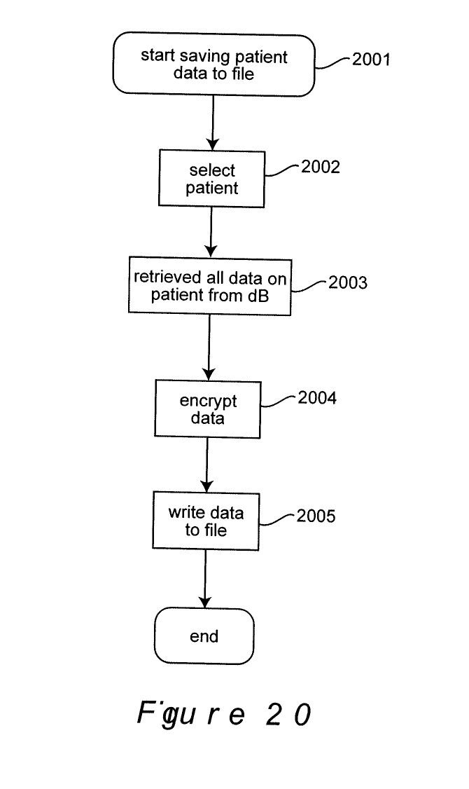

FIG. 19 is a flowchart showing the process implemented for the patient messaging system. The process begins in function block 1901 by selecting the messaging function on the user interface of the patient's handheld device. Incoming messages are retrieved from the system database in function block 1902. The message client is displayed on the patient's handheld device in function block 1903. The patient can send a message or read a message. If the patient chooses to send a message, as determined in decision block 1904, the patient is prompted to compose a message in function block 1905. The composed message is then sent to the system database in function block 1906, from which it is sent to the doctor, as generally indicated in the system diagram of FIG. 1. If the patient chooses to read a message, as determined in decision block 1907, the patient is prompted to select a message in function block 1908. The selected message is displayed in function block 1909. A determination is made in decision block 1910 as to whether the patient wishes to reply. If the patient wishes to reply, the process goes to function block 1905 where the patient composes a reply. Finally, the patient can choose to exit the messaging system as detected by decision block 1911.

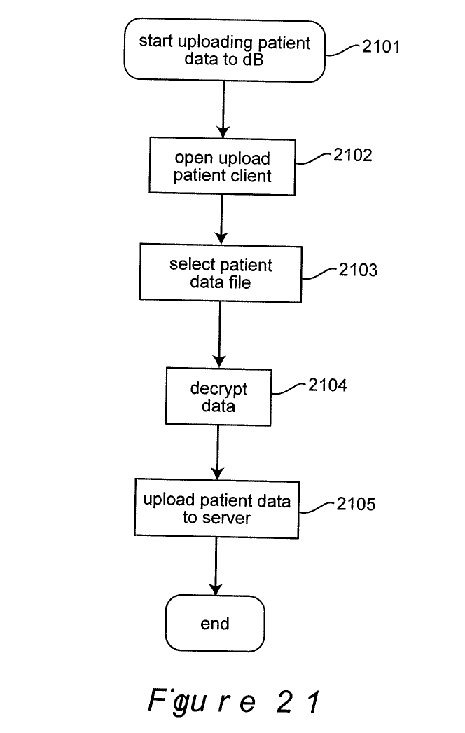

FIG. 20 is a flowchart showing the process of saving patient data to a file. This is a procedure performed using the doctor tablet PC. The process begins in function block 2001 by selecting the save patient data to file from the user interface. The doctor is then prompted to select a patient in function block 2002. As mentioned before, this can be done by the doctor writing in the patient's name or providing a list of patient names from which a name can be selected. Once a patient has been selected, all the data on that patient is retrieved from the system database in function block 2003. The retrieved data is encrypted in function block 2004 before it is written to the file in function block 2005.

FIG. 21 is a flowchart showing the process of uploading patient data to the system database. This is done by accessing the patient client (i.e., handheld device). the process is initiated in function block 2101 which begins by opening the upload patient client in function block 2102. The patient data file is selected in function block 2103. This file is encrypted, so the next step is to decrypt the data in function block 2104. The decrypted patient data is then uploaded to the system server in function block 2105.

While the invention has been described in terms of a single preferred embodiment, those skilled in the art will recognize that the invention can be practiced with modification within the spirit and scope of the appended claims.

Having thus described our invention, what we claim as new and desire to secure by Letters Patent is as follows:

1. A patient healthcare and monitoring system facilitating communications between doctors and patients comprising:
a server hosting a database containing patient information including patient medical histories;
at least one doctor client with which a physician can set up patient alerts, surveys and messages and receive reports, messages and patient history, said at least one doctor client communicating with the server; and
a plurality of patient clients with which a patient receives alerts, messages and surveys generated by their physician and transmit alert completions, messages and survey answers, said plurality of patient clients communicating with the server, whereby patients are alerted when to perform prescribed procedures or take prescribed medications and physicians are informed that prescribed procedures have been performed or medications taken and receive answers to surveys which become part of a patient's medical history.

2. The patient healthcare and monitoring system as recited in claim 1, wherein at least one of said doctor client and said patient client is a mobile device.

3. The patient healthcare and monitoring system recited in claim 2, wherein at least one of said doctor client and said patient client is selected from the group consisting of a tablet PC, a Smart phone, a Pocket PC, a personal data assistant, or other mobile devices which include a screen, input mechanism, messaging capabilities, and at least one processor.

4. The patient healthcare and monitoring system recited in claim 1, wherein the doctor client provides a screen for both text and graphic entry for instruction on a procedure to be performed or a medication to be taken, the text and graphic entry being displayed on a patient client after an alert has been accepted at the patient client.

5. The patient healthcare and monitoring system recited in claim 4, wherein the doctor client further provides a screen for generating alerts for a patient to perform a prescribed procedure or take a medication, the alerts being communicated to a patient client at preset times.

6. The patient healthcare and monitoring system recited in claim 5, wherein the screen for generating alerts includes date and time entry graphics allowing a physician to enter a start date and an end date and times of days for alerts.

7. The patient healthcare and monitoring system recited in claim 6, wherein the time entry graphic is in the form of a circular graphic having an outer ring divided into hours of a day and a next adjacent ring divided into minutes of an hour.

8. The patient healthcare and monitoring system recited in claim 6, wherein the time entry graphic is in the form of selection buttons corresponding to a number of occurrences per day, selection of one of the selection buttons opening a corresponding number of time of day windows for entry of times of alerts.

9. The patient healthcare and monitoring system recited in claim 6, wherein the doctor client further provides a screen for the entry of survey questions to be answered by a patient at a preset time after performing a prescribed procedure or taking a prescribed medication.

10. The patient healthcare and monitoring system recited in claim 9, wherein a patient client upon receiving an alert from the server generates a screen which prompts the user to enter an password in order to accept the alert.

11. The patient healthcare and monitoring system recited in claim 10, wherein after accepting an alert by a user of a patient client, the patient client generates a screen with text and graphic illustration of a prescribed procedure or medication.

12. The patient healthcare and monitoring system recited in claim 11, wherein a user is prompted to input an indication that he or she has completed a prescribed procedure or taken a prescribed medication, which indication is communicated to the server.

13. The patient healthcare and monitoring system recited in claim 12, wherein at a preset time after the indication that a prescribed procedure has been completed or a prescribed medication has been taken is communicated to the server, the patient client receives and generates a survey screen which prompts the user to answer one or more questions presented in the survey screen concerning the user.

14. A method for patient healthcare and monitoring which facilitates communications between doctors and patients comprising the steps of:
establishing a server hosted database containing patient information including patient medical histories;
providing at least one doctor client with which a physician can set up patient alerts, surveys and messages and receive reports, messages and patient history;
establishing a communication link between said at least one doctor client and the server;
providing a plurality of patient clients with which a patient receives alerts, messages and surveys generated by their physician and transmit alert completions, messages and survey answers; and
establishing communication links between each of said plurality of patient clients and the server, whereby patients are alerted when to perform prescribed procedures or take prescribed medications and physicians are informed that prescribed procedures have been performed or medications taken and receive answers to surveys which become part of a patient's medical history.

15. The method for patient healthcare and monitoring recited in claim 14 wherein the steps of providing a doctor client and providing a plurality of patient clients includes using one or more mobile devices for each of said doctor client and said patient clients.

16. The method for patient healthcare and monitoring recited in claim 15 wherein at least one of said doctor client and said patient client is selected from the group consisting of a tablet PC, a Smart phone, a Pocket PC, a personal data assistant, or other mobile devices which include a screen, input mechanism, messaging capabilities, and at least one processor.

17. The method for patient healthcare and monitoring recited in claim 14, further comprising the steps of:
providing on the doctor client a screen for both text and graphic entry for instruction on a procedure to be performed or a medication to be taken; and
displaying the text and graphic entry on a patient client after an alert has been accepted at the patient client.

18. The method for patient healthcare and monitoring recited in claim 17, further comprising the steps of:
providing on the doctor client a screen for generating alerts for a patient to perform a prescribed procedure or take a medication; and
communicating the alerts to a patient client at preset times.

19. The method for patient healthcare and monitoring recited in claim 17, wherein the screen for generating alerts includes date and time entry graphics allowing a physician to enter a start date and an end date and times of days for alerts.

20. The method for patient healthcare and monitoring recited in claim 19, further comprising the step of generating the time entry graphic in the form of a circular graphic having an outer ring divided into hours of a day and a next adjacent ring divided into minutes of an hour.

21. The method for patient healthcare and monitoring recited in claim 19, further comprising the step of generating the time entry graphic in the form of selection buttons corresponding to a number of occurrences per day, selection of one of the selection buttons opening a corresponding number of time of day windows for entry of times of alerts.

22. The method for patient healthcare and monitoring recited in claim 19, further comprising the step of providing on the doctor client a screen for the entry of survey questions to be answered by a patient at a preset time after performing a prescribed procedure or taking a prescribed medication.

23. The method for patient healthcare and monitoring recited in claim 22, further comprising the steps of:
transmitting an alert from the server to a patient client; and
upon receiving an alert from the server by the patient client, generating a screen which prompts the user to enter an password in order to accept the alert.

24. The method for patient healthcare and monitoring recited in claim 23, further comprising the step of generating a screen by the patient client with text and graphic illustration of a prescribed procedure or medication after accepting an alert by a user of a patient client.

25. The method for patient healthcare and monitoring recited in claim 23, further comprising the steps of:
prompting a user to input an indication that he or she has completed a prescribed procedure or taken a prescribed medication; and
communicating the indication input by the user to the server.

26. The method for patient healthcare and monitoring recited in claim 25, further comprising the steps of:
at a preset time after the indication that a prescribed procedure has been completed or a prescribed medication has been taken is communicated to the server, sending by the server to the patient client a survey to be completed by the user;
generating by the patient client a survey screen upon receiving the survey sent by the server; and
prompting by the patient client the user to answer one or more questions of the survey concerning the user.
